(19) United States
(12) Patent Application Publication
(10) Pub. No.: US 2008/0113420 A1
(43) Pub. Date: May 15, 2008

(54) Electrocompetent cells prepackaged for electroporation

(75) Inventor: Stephen P. Kulisch, Pleasant Hill, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Bio-Rad Laboratories, Inc., Hercules, CA (US)

(21) Appl. No.: 11/983,701

(22) Filed: Nov. 9, 2007

Related U.S. Application Data
(63) Continuation of application No. 11/126,491, filed on May 10, 2005, now abandoned.
(60) Provisional application No. 60/570,846, filed on May 12, 2004.

(51) Int. Cl. C12N 13/00; C12M 1/42

(52) U.S. Cl. 435/173.6; 435/285.2

(57) ABSTRACT

Electrocompetent cells are prepackaged in a sterilized cuvette, and the cell contents frozen, such that electroporation can be performed simply by first thawing the cuvette contents, then electrically connecting the cuvette to a power source suitable for electroporation. The cuvette contains, in addition to the cells, a suspending medium, and optionally also a cryoprotectant for the cells. This invention also resides in a process for preparing electroporation cells for use, or alternatively for shipping or transport, comprising forming a suspension of the cells in a suspending medium preferably comprising a cryoprotectant, placing the cell suspension in a sterilized electroporation cuvette, and quickly freezing the cells while in the cuvette. The cells are then stored, and/or shipped or transported under subzero (Celsius) temperatures for subsequent use.

### CROSS-REFERENCES TO RELATED APPLICATIONS

This application is a continuation of U.S. patent application Ser. No. 11/126,491, filed May 10, 2005, which application claims the priority of U.S. Provisional Patent Application No. 60/570,846, filed May 12, 2004. The entire contents of said provisional application are hereby incorporated herein.

### BACKGROUND OF THE INVENTION

This invention resides in the field of electroporation, and in particular to cellular materials used in electroporation, their preparation, storage and/or shipping.

Methods and materials for the transformation of cells by electroporation are well known and widely published. This use of an electric current to insert nucleic acids and other macromolecules into intact living cells has found considerable utility in the research laboratory and holds promise for major advances in medical therapy and biotechnology in general. The following are examples of published literature describing various methods of electroporation and the apparatus and materials that are used in the practice of these methods. Each of these citations is hereby incorporated herein by reference.

Literature supplied by Bio-Rad Laboratories, Inc. (Hercules, Calif., USA), entitled Gene Pulser Xcell System, Bulletin No. 2750US/EG Rev. B, published in January 2004, sets forth descriptions of apparatus, materials, and methods for electroporation of various kinds of cells, both eukaryotic and prokaryotic. Chassy, B. M., et al., “Transformation of lactobacillus casei by electroporation,” FEMS Microbiol. Lett. 44:173-177 (1987) and Powell, I. B., et al., “A Simple and Rapid Method for Genetic Transformation of Lactic Streptococci by Electroporation,” Appl. Environ. Microbiol. 54(3): 655-660 (March 1988) disclose the use of the Bio-Rad Gene Pulser different transformations. Specialized electroporation cuvettes are disclosed by Potter, H., et al., “Enhancer-dependent expression of human kappa immunoglobulin genes introduced into mouse pre-B lymphocytes by electroporation,” Proc. Natl. Acad. Sci. USA 81: 7161-7165 (1984). The electroporation of polyethylene glycol-treated Bacillus protoplasts is described by Shivarova, N., et al., “Microbiological implications of electric field effects. VII. Stimulation of plasmid transformation of protoplasts by electric field pulses,” Zeitschrift Allge. Mikro. 23:595-599 (1983). The transformation of Campylobacter jejuni is described by Miller, J. F., et al., “High voltage electroporation of bacteria: genetic transformation of Campylobacter jejuni with plasmid DNA,” Proc. Natl. Acad. Sci. USA 85: 856-860 (1988). Further disclosures of electroporation cells, materials, and methods are found in Dower, W. J., U.S. Pat. Nos. 4,910,140, Mar. 20, 1990, and 5,186,800, Feb. 16, 1993, Korenstein, R., et al., U.S. Pat. No. 5,964,726, Oct. 12, 1999; Thompson, J. R., U.S. Pat. No. 5,879,891, Mar. 9, 1999; and Greener, A. L., et al., U.S. Pat. No. 6,586,249, Jul. 1, 2003.

Cells that are intended for transformation by electroporation are known as electrocompetent cells, electrocompetency being achieved by suspending normal cells in a low-conductivity medium to prevent arcing during electroporation. Electrocompetent cells are available commercially and are typically sold in microtubes. To perform electroporation, the user first transfers the cell suspension from the microtube to an empty tube, then adds the nucleic acid or other transformation agent, mixes the suspension to distribute the agent, and transfers the combined suspension to a cuvette equipped with electrodes for electroporation. Alternatively, the transformation agent is added to the electrocompetent cell suspension in the microtube, and the combined suspension is then placed in the cuvette. Potential problems with these methods include inaccuracy in the quantities transferred and contamination from transfer implements and intermediate vessels.

### SUMMARY OF THE INVENTION

This invention resides in electrocompetent cells prepackaged in a sterilized cuvette, the cell contents frozen, such that electroporation can be performed simply by first thawing the cuvette contents, then electrically connecting the cuvette to a power source suitable for electroporation. The cuvette will contain, in addition to the cells, a suspending medium, and typically also a cryoprotectant for the cells.

This invention also resides in a process for preparing electroporation cells for use, or alternatively for shipping or transport, comprising forming a suspension of the cells in a suspending medium preferably comprising a cryoprotectant, placing the cell suspension in a sterilized electroporation cuvette, and quickly freezing the cells while in the cuvette. The cells are then stored and/or shipped or transported under subzero (Celsius) temperatures for subsequent use.

### DETAILED DESCRIPTION OF THE INVENTION AND PREFERRED EMBODIMENTS

Cuvettes suitable for use in the practice of this invention are any vessels in which electroporation can be performed. Cuvettes of greatest interest are those that fit into automated electroporation apparatus and that contain the electrical connections necessary for passing a current through the cell suspension. Suitable materials of construction are any materials that are electrically insulating, inert to the cell suspension, and able to withstand strong electrical fields and any other conditions that might be encountered in a typical electroporation procedure. Glass, ceramic, and clear plastic such as polycarbonate are examples of suitable materials. Plastic cuvettes are readily formed by molding. Examples of suitable cuvettes are shown in U.S. Pat. No. 5,186,800, referenced above, in which the electrodes are affixed to the interior surface of, or embedded in, the cuvette walls. The spacing between the electrodes is preferably about 5 mm or less, more preferably from about 1 mm to about 4 mm, and most preferably from about 1.0 mm to about 2.0 mm. The electrodes can be of any configuration, although plate or film electrodes or metal strips are preferred for their ability to produce an electric current over a relatively broad area. Common electrically conductive metals that are corrosion resistant are preferred. Examples are aluminum, silver, gold, and alloys of these metals. The electrode area is preferably from about 5 mm² to about 10 cm², most preferably from about 10 mm² to about 2 cm². The size of the cuvette will preferably be such that the volume between the electrodes, i.e., the volume of the suspension in which electroporation will occur, will range from about 1 μL to about 1 mL, more preferably from about 20 μL to about 500 μL, and most preferably from about 25 μL to about 150 μL.

While the above parameters are merely illustrative, cuvettes can be used that are designed especially for electroporators that are commercially available. Examples of such electroporators are the GENE PULSER® Xcell microbial system, the GENE PULSER® Xcell eukaryotic system, the GENE PULSER® Xcell total system, and the MICROPULSER® Electroporator, all of Bio-Rad Laboratories, Hercules, Calif., USA, the EPPENDORF® Electroporator 2510, the MULTIPORATOR® of Brinkmann Industries, Inc., Westbury, N.Y., USA, the ECM® 2001, ECM® 399, ECM® 630, and ECM® 830 Electroporator Systems, all of Harvard Apparatus Inc., BTX Instrument Division, Holliston, Mass., USA, the NUCLEOFECTOR™ Device of Amaxa Biosystems, Gaithersburg, Md., USA, the CELLJECT UNO, CELLJECT DUO, and CELLJECT PRO, all of Thermo Electron Corporation, Gormley, Ontario, Canada, and THE CLONING GUN™ (BactoZapper™) and THE CLONING GUN™ (MammoZapper™) of Tritech Research, Inc., Los Angeles, Calif., USA. Sterilization of the cuvette is achieved by conventional means such as gamma or ultraviolet irradiation, for example.

Electrocompetent cells for use in the present invention can be prepared by methods known in the electroporation art. A typical preparation method will begin by growing cell cultures to a preselected cell density where the cells are still rapidly dividing. The cells are then harvested by centrifugation or filtration, and then washed, preferably with water or with a low conductivity medium, to lower the quantity of salts present so that when the cells are ultimately suspended in a suspending medium, the electrical conductivity of the suspension will be low enough to prevent arcing in the electroporator. The final cell density in the suspension can vary, although best results in most cases will be achieved with cell concentrations in the range of from about 5×10⁹ to about 5×10¹⁰ cells/mL. The salt concentration in the suspension is preferably low enough that the electrical resistance is about 1,000Ω or above, and most preferably about 5,000Ω or above.

The suspending medium will preferably contain a cryoprotectant to preserve the cells upon freezing. Examples of cryoprotectants are glycerol, polyethylene glycol, polyvinylpyrrolidone, and sugars or sugar derivatives (such as sugar alcohols) beyond those listed above. Examples of sugars and sugar derivatives are trioses such as glyceraldehydes, tetroses such as erythrose and threose, pentoses such as arabinose, xylose, ribose, and lyxose, hexoses such as glucose, mannose, galactose, idose, gulose, altrose, alose and talose, disaccharides such as sucrose, lactose, trehalose, maltose, cellobiose, and gentiobiose, trisaccharides such as raffinose, and oligosaccharides such as amylase, amylopectin, and glycogen.

The cells contained in the cuvette for transformation can be either prokaryotic or eukaryotic. Prokaryotic cells include both grain-positive and gram-negative bacterial cells. Examples of gram-positive bacteria that can be included in the cuvette are Micrococcaceae such as Staphylococcus, Micrococcus, and Sarcina, Streptocacceae such as streptococcus and Leuconostoccus, Lactobacillaceae such as Lactobacillus, Propionibacteriaceae such as Propionibacterium, Corynebacterium, Listeria, and Erysipelothrix, and Baccilaceae such as Bacillus and Clostridium. Examples of gram-negative bacteria are Enterobacteriaceae such as Escherichia, Erwinia, Shigella, Salmonella, Proteus and Yersinia, Bruncellaceae such as Brucella, Bordetella, Pasteurella, and Hemophilus, Azobacteraceae such as Azotobacter, Rhizobiaceae such as Rhizobium, Nitrobacteriaceae such as Nitrosomonas, Nitrobacter, and Thiobacillus, Psuedomonadaceae such as Pseudomonas and Acetobacter, Spirillaceae such as Photobacterium, Zymonomas, Aermona, Vibrio, Desulfovibrio, and Spirilium, and Actinomycetales such as Mycobacterium, Actinomyces, Norcardia, and Streptomyces. Examples of eukaryotic cells are intact animal cells, including mammalian cells, and plant protoplasts.

Once the cell suspension is placed in the sterilized cuvette, the suspension can be frozen in the cuvette by cooling to temperatures below 0° C., and stored indefinitely at such temperatures until ready for use. Typical storage temperatures can range from about −100° C. to about −25° C., preferably at least about −70° C. The freezing is carried out relatively quickly, typically over about 5 minutes. Prior to use, the cuvette and contents will be warmed, preferably at a slow rate, to the temperature at which electroporation will be performed. The cuvette may for example be placed on wet ice (at atmospheric pressure) until equilibrated to the temperature of the ice, and then warmed further. The cell transforming agent can then be added to the cuvette by a conventional transfer implement, and the cuvette is then placed in the electroporator. The cells may be frozen in electroporation cuvettes of different sizes. For example, 0.04 ml or 0.08 ml of the competent cells could be frozen in 0.2 and 0.4 cm gap cuvettes, respectively.

The selection of the transforming agent is an entirely independent choice, and is not limited by the pre-packaged character of the cell suspension in the cuvette. Examples of transforming agents are nucleic acids and other macromolecules such as proteins, enzymes, antibodies, hormones, and carbohydrates, as well as relatively small molecules such as drugs, dyes, labeled nucleotides, and amino acids. Nucleic acids include DNA and RNA, in linear or circular form.

### EXAMPLE

The following is an example of the preparation and use of this invention. However, the invention is not limited thereto.

A sample of E. Coli cells suitable for electroporation is grown using conditions suitable for that purpose. The cells are washed, concentrated, and suspended in a mixture of water and glycerol to give a compositions as follows:

| Component | Weight % |
|---|---|
| Escherichia coli (˜1 × 10¹¹ cells/ml) | 0.001 |
| Glycerol | 10 |
| Water | 89.999 |

Then, 0.2 μL of the mixture is positioned midway between the top and bottom electrodes of a sterile 0.1 cm electroporation cuvette. The cuvette and its contents are frozen to about −70° C. using an ethanol-dry ice bath, for about 5 minutes, and then kept stored at temperatures less than −70° C. until they are to be used or shipped. A similar procedure can be used for a larger sample and cuvette, e.g. 0.4 μL in a sterile 0.2 cm gap electroporation cuvette.

A sample of electroporation cells that had been frozen in the cuvette in this manner was tested in comparison with a sample of similar cells that had been frozen in a vial. Both samples had been stored for about 3-6 months, and shipped frozen from Maryland to California.

The two cell samples were placed in a bucket of wet ice and allowed to thaw. Once thawed, a control plasmid pUC19 was mixed with each sample. The sample in the vial was then transferred to a cuvette. Both cuvettes were placed into an electroporator and pulsed using the manufacturer's recommended conditions. Then 1.0 mL of SOC (Super Optimal Catabolite media) was placed in each cuvette to assist the cells recover after exposure to the current. The volume of each was then removed to a sterile 17×100 mm polypropylene tube and incubated at 37° C. for 1 hour with shaking at 225-250 rpm. The contents of each tube were then diluted 1:100 with SOC and spread onto LB plates containing 100 μg ampicillin. Transformation efficiencies were determined. The sample that had been frozen in the cuvette performed at the same level as the sample that had been frozen in the vial.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A sterilized electroporation cell cuvette containing frozen electrocompetent cells.

2. A cuvette according to claim 1 further containing a suspension medium.

3. A cuvette according to claim 1 further containing a cryoprotectant.

4. A cuvette according to claim 1 that is suitable for use in an automated electroporation apparatus.

5. A cuvette according to claim 1 having electrodes affixed to the interior surface of, or embedded in the walls of, the cuvette.

6. A cuvette according to claim 3 in which the cryoprotectant is selected from sugars, sugar derivatives, glycerol, polyethylene glycol and polyvinylpyrrolidone

7. A cuvette according to claim 6 in which the cryoprotectant is glycerol.

8. A cuvette according to claim 1 in which the cuvette contents have been frozen at a temperature of from about −25 to about −100° C.

9. A cuvette according to claim 1 in which the cuvette contents have been frozen at a temperature of about −70° C.

10. A process for preparing electroporation cells for use comprising
(a) forming a suspension of the cells in a suspending medium optionally comprising a cryoprotectant,
(b) placing the cell suspension in a sterilized electroporation cuvette, and
(c) quickly freezing the cells in the cuvette.

11. A process according to claim 10 in which a cryoprotectant is included in the suspending medium.

12. A process according to claim 11 in which the cryoprotectant is selected from sugars, sugar derivatives, glycerol, polyethylene glycol and polyvinylpyrrolidone.

13. A process according to claim 11 in which the cryoprotectant is glycerol.

14. A process according to claim 10 in which the cuvette contents are frozen at a temperature of from about −25 to about −100° C.

15. A process according to claim 10 in which the cuvette contents are frozen at a temperature of about −70° C.

16. A process according to claim 10 in which step (c) is carried out for about 5 minutes.

17. A process according to claim 10 further comprising packing and shipping the cuvettes in the frozen state, to a receiving location.

18. A process according to claim 17 further comprising thawing the frozen cuvettes at the receiving location and utilizing the thawed cells in an electroporation process.
